(19) United States
(12) Patent Application Publication
(10) Pub. No.: US 2007/0142826 A1
(43) Pub. Date: Jun. 21, 2007

(54) Modification of laser ablation treatment prescription using corneal mechanical properties and associated methods

(76) Inventor: Alex Sacharoff, Oviedo, FL (US)

Correspondence Address: ALCON, IP LEGAL, TB4-8, 6201 SOUTH FREEWAY, FORT WORTH, TX, 76134, US

(21) Appl. No.: 11/303,465

(22) Filed: Dec. 16, 2005

(51) Int. Cl. A61F 9/008

(52) U.S. Cl. 606/10; 606/5

(57) ABSTRACT

A method of the present invention is directed to improving an outcome of a surgical procedure on a patient cornea. The method includes the steps of measuring a mechanical parameter of a cornea of an eye and determining a laser-surgery prescription for the cornea to improve a visual parameter therefor. The prescription is then adjusted based upon the measured mechanical parameter of the cornea.

### FIELD OF THE INVENTION

The invention relates generally to laser surgical systems and methods, and more particularly to such systems and methods for achieving corneal ablation.

### BACKGROUND OF THE INVENTION

The use of lasers to erode all or a portion of a workpiece's surface is known in the art. In the field of ophthalmic medicine, modification of corneal curvature is known to be accomplished using ultraviolet or infrared lasers. The procedure has been referred to as “corneal sculpting.”

Prior to the application of the sculpting laser, a lenticular flap of tissue is lifted that can have a thickness of 100-200 μm. A laser beam is then delivered onto the exposed stromal surface to achieve a desired correction using a predetermined prescription, and the flap is replaced.

Although this surgery is very successful for most patients, occasionally a second procedure must be undertaken in order to enhance the original refractive result. The first procedure will have slightly weakened the cornea, owing to the creation of the flap itself. Also, there is a limit to the amount of ablation that can be performed, since an acceptable amount of corneal structure must remain after a second procedure.

Currently the surgeon is not required to measure eye parameters other than corneal thickness and the refractive error. Some surgeons also measure surface topography information, but it is believed that this has little predictive value in determining the outcome for any specific eye with the application of any specific wavefront aberrometer or phoropter-derived ablation profile.

In the performance of any surgical procedure, a certain number of “outliers” with regard to outcomes will occur wherein the result does not appear to be related directly to the process. It may be, for example, that some individuals have corneal tissue that is mechanically different from the norm. In such cases the cornea's response to laser surgery can result in an unexpected or over-correction.

Surgeons develop nomogram adjustments by following the outcomes of a number of patients and looking for trends in the patient population as a whole. It is believed that no measurements have been made of eye properties that could lead to outliers.

Contact and non-contact tonometers are known in the art that are capable of measuring corneal mechanical properties by means of detecting a vibrational recoil response (“corneal hysteresis”). The construction of a biomechanical model of the cornea using finite-element analytical methods is also known in the art.

### SUMMARY OF THE INVENTION

The present invention is useful for accomplishing surgical procedures, such as, for example, photorefractive keratectomy (PRK), phototherapeutic keratectomy (PTK), and laser in situ keratomileusis (LASIK). It is believed that at least some of the “outliers” in surgical outcomes can be the result of mechanical properties of the cornea that lie outside the norm. An identification of such potential outliers can result in the elimination of such patients from laser surgery, or in the adjustment of the measured prescription in order to take the corneal mechanical properties into account. By practicing the method of the present invention, a collection of data on the underlying structural differences in patient corneas can be used as additional input into a surgeon's nomograms.

A method of the present invention is directed to improving an outcome of a surgical procedure on a patient cornea. The method comprises the steps of measuring a mechanical parameter of a cornea of an eye and determining a laser-surgery prescription for the cornea to improve a visual parameter therefor. The prescription is then adjusted based upon the measured mechanical parameter of the cornea.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWING

FIG. 1 is a schematic for the system of the present invention.

FIG. 2 is a flowchart of an embodiment of a method of the present invention.

### DETAILED DESCRIPTION OF THE PREFERRED EMBODIMENTS

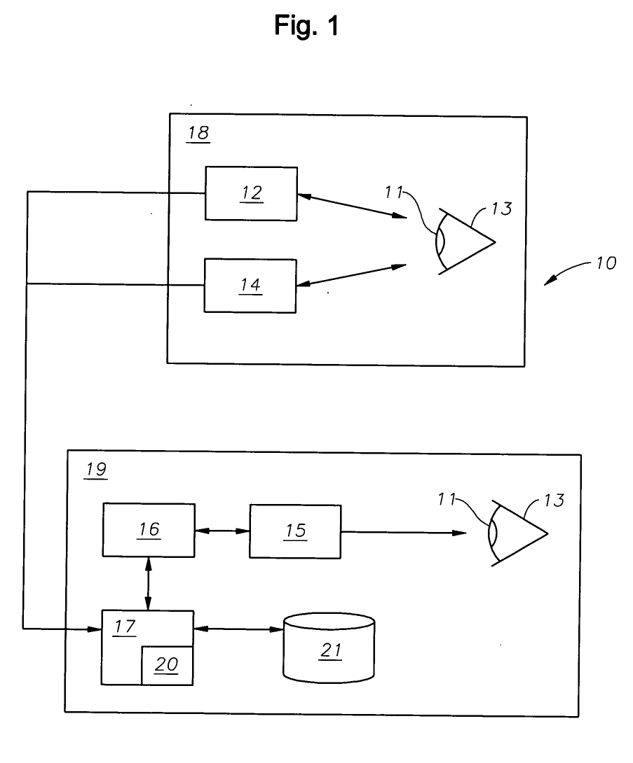

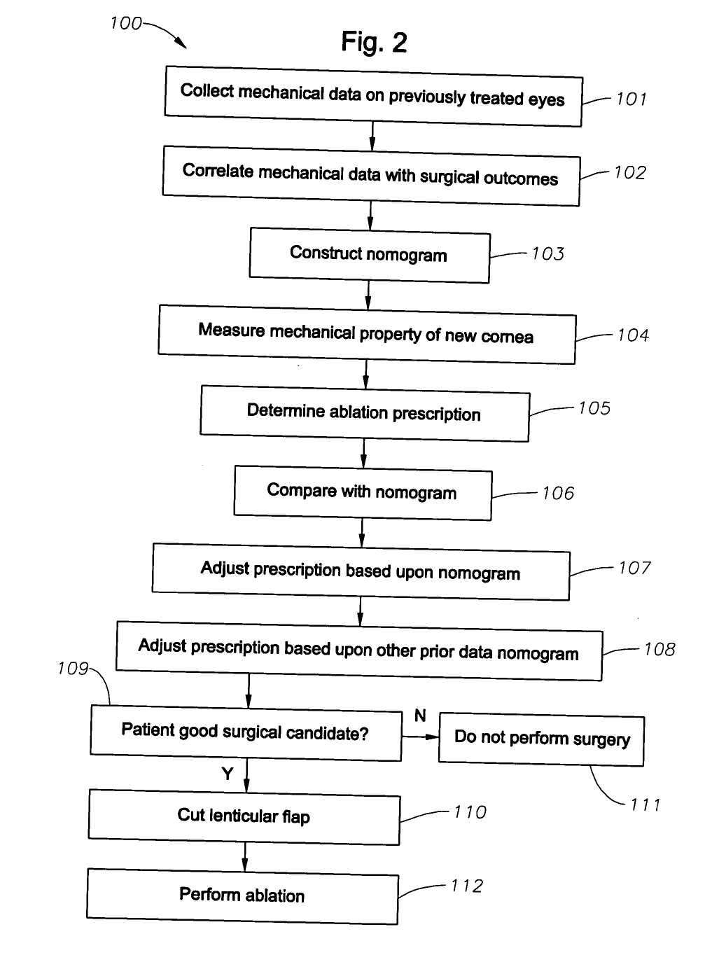

The present invention will now be described with reference to FIGS. 1 and 2. One aspect of the present invention is directed to a system 10 for performing a surgical procedure on a patient cornea 11 (FIG. 1). The system 10 comprises a device 12 for measuring a mechanical parameter of a cornea 11 of an eye 13. Such a device 12 may comprise, but is not intended to be limited to, a device for measuring: a minimum and maximum corneal diameter, an angle between the minimum and the maximum diameters, a thickness of the cornea at a center thereof, a thickness of the cornea at a plurality of locations in spaced relation from the cornea center, an intraocular pressure of the cornea, an indent parameter on the cornea from a force applied thereto, and an acoustic waveform detected following an application to the cornea of an impulsive force thereto.

The system 10 further comprises a device 14 for determining a laser-surgery prescription for the cornea 11 to improve a visual parameter therefor. Such a device 14 may comprise, for example, a wavefront aberrometer, although this is not intended as a limitation.

The system 10 additionally comprises a treatment laser 15, a laser controller 16, and a processor 17 in signal communication with the laser controller 16. Typically the aberrometer 14 and the treatment laser 15 will be situated in different locations 18,19, as the wavefront measurements are usually performed prior to performing laser ablation. However, these devices 14,15 may also be collocated in some embodiments. Resident on the processor 17 is a software package 20 having code segments for carrying out the calculations to be described in the following.

An exemplary embodiment of a method 100 of the present invention for performing a surgical procedure on a patient cornea comprises the steps of collecting mechanical parameter data on a plurality of previously treated eyes (block 101) and performing a statistical analysis to determine a correlation between the collected mechanical parameter data and patient surgical outcomes (block 102). This analysis is used to construct a nomogram for use in subsequent cases (block 103), which is stored, for example, in a database 21 that is accessible by the processor 17.

A mechanical parameter of the cornea is measured (block 104), as well as a laser-surgery prescription for the cornea to improve a visual parameter therefor (block 105). The mechanical parameter is compared with the nomogram (block 106), and, if appropriate, the prescription is adjusted based upon the measured mechanical parameter of the cornea (block 107). The prescription may also be adjusted based upon previously determined nomograms that can be, for example, site- and/or device-dependent (block 108).

The comparison of block 106 is used to make a determination as to patient candidacy for a laser-surgery procedure (block 109). If the patient is not a candidate, the procedure is not performed (block 111). If the patient is determined to be a candidate for the procedure, a lenticular flap is cut in the cornea (block 110), and the treatment laser 15 is controlled to ablate corneal tissue according to the adjusted prescription (block 112).

The present invention is thus capable of identifying potential “outliers” and eliminating them from the pool of potential surgical candidates, and also of adjusting prescriptions based upon a nomogram constructed from an analysis of mechanical data on a plurality of previously examined eyes, thereby improving treatment outcomes.

Although the invention has been described relative to specific embodiments thereof, there are numerous variations and modifications that will be readily apparent to those skilled in the art in the light of the above teachings. It is therefore to be understood that, within the scope of the appended claims, the invention may be practiced other than as specifically described.

What is claimed is:

1. A method for improving an outcome of a surgical procedure on a patient cornea comprising the steps of:
measuring a mechanical parameter of a cornea of an eye;
determining a laser-surgery prescription for the cornea to improve a visual parameter therefor; and
adjusting the prescription based upon the measured mechanical parameter of the cornea.

2. The method recited in claim 1, wherein the mechanical parameter is selected from a group consisting of a minimum and maximum corneal diameter, an angle between the minimum and the maximum diameters, a thickness of the cornea at a center thereof, a thickness of the cornea at a plurality of locations in spaced relation from the cornea center, an intraocular pressure of the cornea, an indent parameter on the cornea from a force applied thereto, and an acoustic waveform detected following an application to the cornea of an impulsive force thereto.

3. The method recited in claim 1, wherein the prescription-determining step comprises collecting wavefront aberration data on the eye.

4. The method recited in claim 1, wherein the adjusting step comprises correlating the measured mechanical parameter with collected data on previously measured corneas.

5. The method recited in claim 1, further comprising the steps, prior to the adjusting step, of collecting mechanical parameter data on a plurality of eyes and performing a statistical analysis to determine a correlation between the collected mechanical parameter data and patient surgical outcomes, and wherein the adjusting step comprises comparing the measured mechanical parameter with the determined correlation.

6. The method recited in claim 1, further comprising the step, following the determining step, of revising the prescription based upon previously determined nomogram data.

7. A method for performing a surgical procedure on a patient cornea comprising the steps of:
measuring a mechanical parameter of a cornea of an eye;
determining a laser-surgery prescription for the cornea to improve a visual parameter therefor;
adjusting the prescription based upon the measured mechanical parameter of the cornea;
making a determination as to patient candidacy for a laser-surgery procedure;
if the patient is determined to be a candidate for the procedure, cutting a lenticular flap in the cornea; and
controlling a laser to ablate corneal tissue according to the adjusted prescription.

8. The method recited in claim 7, wherein the mechanical parameter is selected from a group consisting of a minimum and maximum corneal diameter, an angle between the minimum and the maximum diameters, a thickness of the cornea at a center thereof, a thickness of the cornea at a plurality of locations in spaced relation from the cornea center, an intraocular pressure of the cornea, an indent parameter on the cornea from a force applied thereto, and an acoustic waveform detected following an application to the cornea of an impulsive force thereto.

9. The method recited in claim 7, wherein the prescription-determining step comprises collecting wavefront aberration data on the eye.

10. The method recited in claim 7, wherein the adjusting step comprises correlating the measured mechanical parameter with collected data on previously measured corneas.

11. The method recited in claim 7, further comprising the steps, prior to the adjusting step, of collecting mechanical parameter data on a plurality of eyes and performing a statistical analysis to determine a correlation between the collected mechanical parameter data and patient surgical outcomes, and wherein the adjusting step comprises comparing the measured mechanical parameter with the determined correlation.

12. The method recited in claim 7, further comprising the step, following the determining step, of revising the prescription based upon previously determined nomogram data.

13. A system for improving an outcome of a surgical procedure on a patient cornea comprising a software package resident on a computer-readable medium, the software package comprising code segments adapted to:
receive measurement data relating to a mechanical parameter of a cornea of an eye;
receive a laser-surgery prescription for the cornea to improve a visual parameter therefor; and
calculate an adjustment to the prescription based upon the measured mechanical parameter of the cornea.

14. The system recited in claim 13, wherein the mechanical parameter is selected from a group consisting of a minimum and maximum corneal diameter, an angle between the minimum and the maximum diameters, a thickness of the cornea at a center thereof, a thickness of the cornea at a plurality of locations in spaced relation from the cornea center, an intraocular pressure of the cornea, an indent parameter on the cornea from a force applied thereto, and an acoustic waveform detected following an application to the cornea of an impulsive force thereto.

15. The system recited in claim 13, wherein the prescription comprises an ablation profile based upon wavefront aberration data on the eye.

16. The system recited in claim 13, wherein the adjustment-calculating code segment comprises a code segment for correlating the measured mechanical parameter with collected data on previously measured corneas.

17. The system recited in claim 13, wherein the software package further comprises code segments for, prior to the adjusting step, receiving mechanical parameter data on a plurality of eyes and performing a statistical analysis to determine a correlation between the collected mechanical parameter data and patient surgical outcomes, and wherein the adjusting code segment comprises a code segment for comparing the measured mechanical parameter with the determined correlation.

18. The system recited in claim 13, wherein the software package further comprises a code segment for revising the prescription based upon previously determined nomogram data.

19. A system for performing a surgical procedure on a patient cornea comprising:
a device for measuring a mechanical parameter of a cornea of an eye;
a device for determining a laser-surgery prescription for the cornea to improve a visual parameter therefor;
a software package comprising codes segments for:
adjusting the prescription based upon the measured mechanical parameter of the cornea; and
making a determination as to patient candidacy for a laser-surgery procedure;
if the patient is determined to be a candidate for the procedure, a cutter for making a lenticular flap in the cornea;
an ablation laser; and
means for controlling the laser to ablate corneal tissue according to the adjusted prescription.

20. The system recited in claim 19, wherein the mechanical parameter measuring device is selected from a group consisting of devices for measuring: a minimum and maximum corneal diameter, an angle between the minimum and the maximum diameters, a thickness of the cornea at a center thereof, a thickness of the cornea at a plurality of locations in spaced relation from the cornea center, an intraocular pressure of the cornea, an indent parameter on the cornea from a force applied thereto, and an acoustic waveform detected following an application to the cornea of an impulsive force thereto.

21. The system recited in claim 19, wherein the prescription-determining device comprises a device for collecting wavefront aberration data on the eye.

22. The system recited in claim 19, wherein the adjusting code segment comprises a code segment for correlating the measured mechanical parameter with collected data on previously measured corneas.

23. The system recited in claim 19, wherein the software package further comprises code segments for receiving mechanical parameter data on a plurality of eyes and performing a statistical analysis to determine a correlation between the collected mechanical parameter data and patient surgical outcomes, and wherein the adjusting code segment is for comparing the measured mechanical parameter with the determined correlation.

24. The system recited in claim 19, wherein the software package further comprises a code segment for revising the prescription based upon previously determined nomogram data.
